(19) United States
(12) Patent Application Publication
(10) Pub. No.: US 2021/0230662 A1
(43) Pub. Date: Jul. 29, 2021

(54) Methods for the Treatment of Solid Tumor Cancers Using Illudins and Biomarkers

(71) Applicant: Lantern Pharma Inc., Dallas, TX (US)

(72) Inventors: Aditya Kulkarni, Dallas, TX (US); Yuvanesh Vedaraju, Dallas, TX (US); Umesh Kathad, Dallas, TX (US); Arun Asaithambi, Dallas, TX (US)

(21) Appl. No.: 17/230,821

(22) Filed: Apr. 14, 2021

Related U.S. Application Data
(63) Continuation of application No. PCT/US2019/005603, filed on Oct. 14, 2019.
(60) Provisional application No. 62/745,382, filed on Oct. 14, 2018.

(51) Int. Cl. C12Q 1/26; C12Q 1/42; G01N 33/68; A61K 31/17

(52) U.S. Cl. CPC C12Q 1/26; C12Q 1/42; G01N 33/6893; G01N 2333/90206; G01N 2333/916; G01N 2333/90245; A61K 31/17

(57) ABSTRACT

Methods for determining the likelihood that a subject suffering from a solid tumor cancer will benefit from treatment with an illudin are disclosed herein. Further, there are also methods for treatment based on such determination. In several embodiments, markers prostaglandin reductase 1 (PTGR1), Protein Tyrosine Phosphatase Non-Receptor Type 14 (PTPN14), Aspartate Beta-Hydroxylase (ASPH) together with one or more genes or alone may be used to enhance or guide treatment with an illudin. In certain embodiments, the protein or gene may be expressed or methylated.

### CROSS-REFERENCE TO RELATED APPLICATION

This application claims the benefit of U.S. Provisional Patent Application No. 62/745,382, filed Oct. 14, 2018, which is incorporated by reference herein in its entirety.

### TECHNICAL FIELD

This invention relates to a marker or markers for use in determination of the sensitivity of a cancer patient to an anti-cancer agents (e.g., illudins) to be administered thereto, which marker can determine whether or not the cancer of the patient has a therapeutic response to the anti-cancer agent, and to application of the marker.

### BACKGROUND

Illudins have been chemically modified and used to treat cancer. Irofulven, an illudin, is a chemically modified version of the fungal toxin Illudin S. It is a DNA-alkylating agent that has an unusual mechanism. This illudin is a DNA and protein-damaging agent that targets rapidly dividing cells of malignant tumors. It enters the tumor cells where it interferes with DNA replication and cell division by binding to DNA and to protein targets. This leads the tumor cells to shut down and consequently die (apoptosis). A study determined that at certain doses tumor cells were highly susceptible to Irofulven's dual damaging activity while normal cells showed only marginal response to the cell-killing drug. Not all solid cancers are sensitive to illudins and methods to determine subjects that are sensitive to illudins has been elusive.

Despite recent developments in diagnostic and therapeutic strategies for subjects with solid tumors especially prostate, ovarian, liver and kidney cancers, there are a number of critical knowledge gaps in relation to their screening and treatment. There is insufficient knowledge of patient characteristics, including genetic profiles, for optimal stratification of subjects into response groups at the time of diagnosis. There is also insufficient knowledge of the risk factors for developing or dying from these cancers and a lack of effective implementation of real-world evidence into clinical practice. This lack of knowledge means that predicting which subjects will have the best outcomes with specific treatments is suboptimal. In addition, current predictions of which subjects may be harmed by unnecessary or inappropriate treatment or managed safely without treatment remain poor.

Accordingly, there is a need for a method for treating and determining whether a subject will be or is more or less likely to respond to an illudin treatment. It is to this need, among others, that this application is directed.

### SUMMARY

This application relates to methods for determining the likelihood that a subject suffering from a solid tumor cancer will benefit from treatment with an illudin. Further, there are also methods for treatment based on such determination. In several embodiments, markers prostaglandin reductase 1 (PTGR1), Protein Tyrosine Phosphatase Non-Receptor Type 14 (PTPN14), Aspartate Beta-Hydroxylase (ASPH) together with one or more genes or alone are used in methods. In certain embodiments, the protein or gene may be expressed or methylated.

A method of treating solid tumor cancer with a targeted drug therapy comprising:

identifying a patient having cancer sensitive to treatment with a compound of the formula:

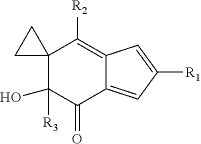

wherein R1, R2 and R3 are independently (C1-C4) alkyl, methyl, or hydroxyl.

### Definitions

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a condition, or one or more of the symptoms associated with the condition; or alleviating or eradicating the cause(s) of the condition itself.

The terms “manage,” “managing,” and “management” encompass preventing the recurrence of the specified disease, disorder, or condition in a patient who has already suffered from the disease, disorder, or condition, and/or lengthening the time that a patient who has suffered from the disease, disorder, or condition remains in remission. The terms encompass modulating the threshold, development and/or duration of the disease, disorder, or condition, or changing the way that a patient responds to the disease, disorder, or condition.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “sensitivity” and “sensitive” when made in reference to treatment with compound is a relative term which refers to the degree of effectiveness of the compound in lessening or decreasing the progress of the disease, disorder, or condition being treated. For example, the terms “increased sensitivity” or “sensitive to treatment” when used in reference to treatment of a disease, disorder, or condition in connection with a compound refers to an increase of at least 3%, in particular at least 5%, or more, in the effectiveness of the treatment.

As used herein, and unless otherwise specified, the term “therapeutically effective amount” of a compound is an amount sufficient to provide a therapeutic benefit in the treatment or management of a disease, disorder, or condition, or to delay or minimize one or more symptoms associated with the presence of the disease, disorder, or condition. A therapeutically effective amount of a compound means an amount of therapeutic agent, alone or in combination with other therapies, which provides a therapeutic benefit in the treatment or management of the disease, disorder, or condition. The term “therapeutically effective amount” can encompass an amount that improves overall therapy, reduces or avoids symptoms or causes of the disease, disorder, or condition, or enhances the therapeutic efficacy of another therapeutic agent.

As used herein, an “effective patient response” refers to any increase in the therapeutic benefit to the patient. An “effective patient response” can be, for example, a 5%, 7.5%, 10%, 15%, 20%, 25%, 30%, 35%, 40%, 45%, 50%, 60%, 70%, 75%, 80%, 90%, or 100% decrease in the rate of progress of the disease, disorder, or condition. An “effective patient response” can be, for example, a 5%, 7.5%, 10%, 15%, 20%, 25%, 30%, 35%, 40%, 45%, 50%, 60%, 70%, 75%, 80%, 90%, or 100% decrease in the physical symptoms of a disease, disorder, or condition. An “effective patient response” can also be, for example, a 5%, 7.5%, 10%, 15%, 20%, 25%, 30%, 35%, 40%, 45%, 50%, 60%, 70%, 75%, 80%, 90%, 100%, 120%, 140%, 150%, 170%, 180%, 190%, 200%, or more increase in the response of the patient, as measured by any suitable means, such as gene expression, cell counts, assay results, etc.

An improvement in the disease, disorder, or condition can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable disease, disorder, or condition burden (i.e., the number of malignant cells present in the subject, or the measured bulk of tumor masses or the quantity of abnormal monoclonal protein) in the absence of new lesions. The term “treatment” contemplates both a complete and a partial response.

The term “refractory or resistant” refers to a circumstance where patients, even after intensive treatment, do not respond to treatment. For example, patients may have residual cancer cells (e.g., leukemia or lymphoma cells) in their lymphatic system, blood and/or blood forming tissues (e.g., marrow).

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

The terms “isolated” and “purified” refer to isolation of a substance (such as DNA/mRNA or protein) such that the substance comprises a substantial portion of the sample in which it resides, i.e. greater than the substance is typically found in its natural or un-isolated state. Typically, a substantial portion of the sample comprises, e.g., greater than 1%, greater than 2%, greater than 5%, greater than 10%, greater than 15%, greater than 20%, greater than 25%, greater than 30%, greater than 40%, greater than 50%, or more, usually up to about 90%-100% of the sample. For example, a sample of isolated DNA/mRNA can typically comprise at least about 1% total mRNA. Techniques for purifying polynucleotides are well known in the art and include, for example, gel electrophoresis, ion-exchange chromatography, affinity chromatography, flow sorting, and sedimentation according to density.

The term “sample” as used herein relates to a material or mixture of materials, typically, although not necessarily, in fluid form, containing one or more components of interest.

“Biological sample” as used herein refers to a sample obtained from a biological subject, including sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Biological samples include but are not limited to whole blood, partially purified blood, PBMCs, tissue biopsies, and the like.

As used herein, and unless otherwise indicated, the term “optically pure” means a composition that comprises one optical isomer of a compound and is substantially free of other isomers of that compound. For example, an optically pure composition of a compound having one chiral center will be substantially free of the opposite enantiomer of the compound. An optically pure composition of a compound having two chiral centers will be substantially free of other diastereomers of the compound. A typical optically pure compound comprises greater than about 80% by weight of one enantiomer of the compound and less than about 20% by weight of other enantiomers of the compound, or greater than about 90% by weight of one enantiomer of the compound and less than about 10% by weight of the other enantiomers of the compound, or greater than about 95% by weight of one enantiomer of the compound and less than about 5% by weight of the other enantiomers of the compound, or greater than about 97% by weight of one enantiomer of the compound and less than about 3% by weight of the other enantiomers of the compound, or greater than about 99% by weight of one enantiomer of the compound and less than about 1% by weight of the other enantiomers of the compound.

As used herein, the term “illudin” includes the compound with formula I:

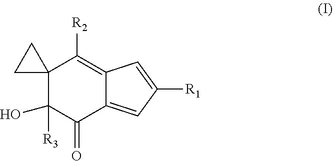

The R₁, R₂ and R₃ are independently (C1-C4) alkyl, methyl, or hydroxyl. The term illudin may include HydroxyMethylAcylfulvene (HMAF, Irofulven), which has the following formula II:

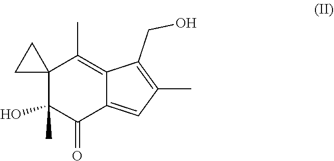 [SMILES: CC1=C(CO)C2=C(C)C3(CC3)[C@@](C)(O)C(=O)C2=C1]

The term illudin also may include (−)-HydroxyUreaMethylAcylfulvene, which has the following formula III

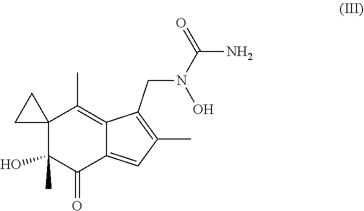 [SMILES: CC1=C(CN(O)C(N)=O)C2=C(C)C3(CC3)[C@@](C)(O)C(=O)C2=C1]

### DETAILED DESCRIPTION

One member of the anti-cancer agent sensitivity determination marker includes gene markers prostaglandin reductase 1 (PTGR1), Protein Tyrosine Phosphatase Non-Receptor Type 14 (PTPN14), Aspartate Beta-Hydroxylase (ASPH) together with one or more genes or alone. In certain embodiments, the protein gene may be expressed or methylated. Prostaglandin reductase 1 (PTGR1) is a highly inducible enzyme with enone reductase activity. The marker PTGRI was correlated with sensitivity to illudin-based treatments or correlated with true responders to illudin-based treatments. The use of the marker, alone or with others, can reduce the gaps in the treatment of cancers (e.g., prostate cancers) by developing predictive biomarker-based screening tests enabling precision medicine-based therapies to patients. The inventors have discovered that detection of solid tumor-associated mutated nucleic acid or protein sequences circulating in subjects with a solid tumor cancer that can accurately determine whether the subject is sensitive to a treatment with an illudin.

In specific embodiments, the DNA of the PTGR1, PTPN14, ASPH genes are methylated. DNA methylation is a process by which methyl groups are added to the DNA molecule. Methylation can change the activity of a DNA segment without changing the sequence. When located in a gene promoter, DNA methylation typically acts to repress gene transcription.

A method of treating or managing solid tumor cancer (e.g., prostate cancer, ovarian cancer, liver cancer, kidney cancer, and thyroid cancer) includes

identifying a patient having cancer sensitive to treatment with an illudin of the formula:

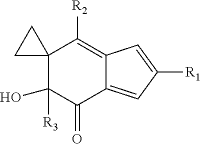

illudin or a pharmaceutically acceptable salt, solvate or stereoisomer thereof; and

administering to the patient a therapeutically effective amount of the illudin.

A method of treating or managing solid tumor treatment includes

identifying a patient having solid tumor cancer sensitive to treatment with an illudin having the formula:

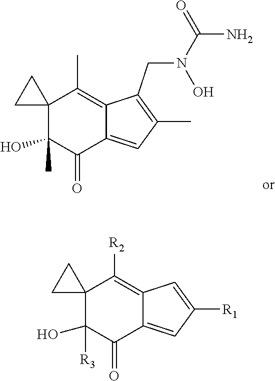

or a pharmaceutically acceptable salt, solvate or stereoisomer thereof; and

administering to the patient a therapeutically effective amount of the compound.

A method for predicting response to treatment in a patient having a solid tumor, comprising:

obtaining a biological sample from the patient;

measuring the level of expression of PTGR1, PTPN14, ASPH or a combination thereof in the biological sample; and

comparing the level of expression in the biological sample to that of a biological sample from a subject not having a solid cancer;

wherein an increased level of the expression in the biological sample from the patient relative to that from the subject not having the solid tumor indicates a likelihood of an effective response to the treatment with a compound of the formula:

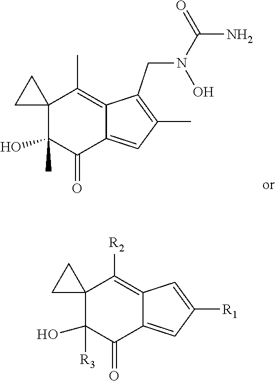

or a pharmaceutically acceptable salt, solvate or stereoisomer thereof.

A method for predicting response to treatment in a patient having a solid tumor, comprising: obtaining a biological sample from the patient;

measuring the level of expression of PTGR1, PTPN14, ASPH or a combination thereof in the biological sample; and

comparing the level of the expression of PTGR1, PTPN14, ASPH or a combination thereof in the biological sample to that of a biological sample from a subject not having a solid tumor;

wherein a decreased level of expression of PTGR1, PTPN14, ASPH or a combination thereof in the biological sample from the patient relative to that from the subject not having a solid tumor indicates a likelihood of an effective response to the treatment with a compound of the formula:

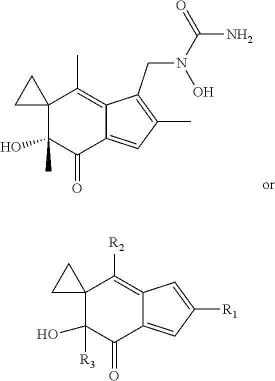

or a pharmaceutically acceptable salt, solvate or stereoisomer thereof.

In one embodiment provided herein is a method of treating or managing solid tumor cancer, comprising: identifying a patient having solid tumor cancer sensitive to treatment with an illudin, or a pharmaceutically acceptable salt; and administering to the patient a therapeutically effective amount of the compound. In certain embodiments, methods provided herein further comprise administering a therapeutically effective amount of an additional active agent, which is illudin.

In one embodiment, the illudin is administered once on day 1 of a 21 day cycle or another cycle.

In another embodiment, the illudin may be administered with one or more second active ingredients. Such active ingredients may be other anti-cancer agents or agents used to treat cancer patients.

As set out above, the solid tumor cancer may be selected from the group consisting of colorectal cancer, pancreatic cancer, primary liver cancers, kidney cancer, ovarian cancer, uterine cancer, lung cancer, breast cancer, prostate cancer, sarcomas, and adipose tissue cancer.

In one embodiment, one or more of the following genes may be used as a marker with PTGR1 to determine sensitivity to an illudin-based anticancer agent: TNFRSF1B, LCK, TAL1, GNG12, S100A10, CD247, ATP1B1, LAMC1, PTPRC, PTPN7, PTPN14, CAPN2, ENAH, LCT, CXCR4, ITGA4, CASP10, HDAC4, NUP210, WWTR1, PFN2, MLF1, CD38, RHOH, ITK, LCP2, HIST1H3B, MYB, IKZF1, PIK3CG, MET, NRF1, TRBC1, PTPRN2, PTK2B, ASPH, NFIB, CNTRL, PRKCQ, MYOF, CTBP2, LMO2, CD3D, TWF1, NCKAP1L, CKAP4, LCP1, CFL2, TJP1, ALDH1A2, TLE3, TNFRSF12A, PRKCB, NFATC3, WWOX, CBFA2T3, ATP2A3, GIPC1, HAUS5, MAP4K1, SDC4, APP, SLC38A5, WAS, and IL2RG.

In another embodiment, one or more of the following gene markers may be used WWTR1, HIST1H3B, ASPH, MYOF, CTBP2, CBFA2T3 and SDC4. In another embodiment, one or more markers were combined with PTGR1 with one, more or all of the following genes with PTGR1: PTPN14, ITGA4, HDAC4, ITK, IKZF1, PIK3CG, NRF1, ASPH, and PRKCQ. In some instances, the sensitivity can approach 100%.

In yet another embodiment, the two markers PTGR1 and SDC4 may be used as the markers.

In the instance where a PTGR1 combination of genes is used, screening of an anti-cancer agent sensitivity enhancer can be performed through employment, as an index, of variation in expression of a PTGR1 combination of genes after exposure to the illudin-based anti-cancer agent. That is, a substance which decreases the PTGR1 combination of genes level before exposure to the illudin-based anti-cancer agent or which promotes variation in the expression or elevates the level after exposure to the illudin-based anti-cancer agent, in vitro or in vivo, enhances the sensitivity to the anti-cancer agent. In addition, in an in vitro case, a substance which promotes variation in the expression or elevates the level after exposure to the anti-cancer agent corresponding to the target cancer cells can serve as a substance which enhances the sensitivity to the illudin-based anti-cancer agent (i.e., anti-cancer agent sensitivity enhancer).

In order to carry out the method for determining sensitivity of a specimen to an anti-cancer agent, a kit containing a protocol for measuring any of the substances present in the specimen is employed. The kit contains a reagent for measuring any of these substances, an indication of an instruction manual for use of the reagent, standards for determining the presence or absence of sensitivity to the illudin-based anti-cancer agent, etc. The standards include (relative) standard levels of these metabolism-related substances, a (relative) high threshold level, a (relative) low threshold level, factors affecting the measurements, the degree of the effects, etc. These substance levels may be set so as to suit the illudin-based anti-cancer agent selected. The sensitivity determination may be performed in the same manner on the basis of the standards.

Screening of an illudin-based anti-cancer agent can be performed by means of the illudin-based anti-cancer agent sensitivity determination marker as an index. That is, a substance which can vary the level of the anti-cancer agent sensitivity determination marker in vitro or in vivo is evaluated as an anti-cancer agent. For example, in an in vitro case, a substance which varies the anti-cancer agent sensitivity determination marker level in various cancer cells after exposure to the substance can serve as an anti-cancer agent. Also, when the anti-cancer agent sensitivity determination marker level in a cancer-bearing animal is varied after administration of a substance thereto, the substance can serve as an anti-cancer agent. If the anti-cancer agent is expected to exhibit a pharmacological effect, the increase in anti-cancer agent sensitivity determination marker level is observed before occurrence of tumor shrinkage or attaining cytocidal effect. Therefore, screening based on the anti-cancer agent sensitivity determination marker level as an index can realize, for a shorter period of time, determination whether or not the test substance serves as a useful anti-cancer agent, whereby efforts and cost involved in the development of anti-cancer agents are greatly expected to be reduced.

When the cancer has no sensitivity to an anti-cancer agent, no pharmacological effect can be expected from the anti-cancer agent. If such a pharmaceutically impotent anti-cancer agent is continuously administered to the patient, the cancer may progress, and side effects may be aggravated. Thus, the anti-cancer agent sensitivity determination marker may be employed not only to determine therapeutic response to the anti-cancer agent, but also to greatly contribute to prevention of aggravation of side effects, which would otherwise be caused by continuous administration of a pharmaceutically inactive anti-cancer agent.

Through employment, in combination, of the thus-obtained anti-cancer agent sensitivity enhancer and an illudin-based anti-cancer agent which is a sensitivity enhancement target of the enhancer, the therapeutic effect of the illudin-based anti-cancer agent is drastically enhanced. The combination of the illudin-based anti-cancer agent sensitivity enhancer and the anti-cancer agent which is a sensitivity enhancement target of the enhancer may be a composition containing both ingredients, or a combined drug of preparations containing individual ingredients.

To assist with monitoring of subjects, it is particularly if the reference value is obtained from the subject, as part of an on-going screening program. Alternatively, the reference value may be obtained from a population of subjects who have no solid tumor cancer.

Cells for testing may be obtained by any method known in the art, including but not limited to as a surgical resection, as a biopsy for example but not limited to a needle biopsy, core biopsy, or aspirate, or collection from a fluid sample, such as blood, urine, cerebral spinal fluid, cystic fluid, etc. Methods of measuring DNA/mRNA include but are not limited to polymerase chain reaction, in situ hybridization, gel electrophoresis, sequence analysis, and microarray analysis or a combination thereof. Methods of measuring protein include but are not limited to mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation, and immunohistochemistry. Antibody arrays or protein chips may also be employed. That an anticancer effect is “likely” to be produced by an agent in a subject means that the subject, in the parameter or parameters being tested (e.g., level of PTGR1, PTPN14 and ASPH mRNA, methylated DNA and/or protein, expression of genes or exons listed in Example 1), is more similar to other subjects in which the agent produces a significant anticancer effect than to other subjects in which the agent does not produce a significant anticancer effect. That an anticancer effect is “unlikely” to be produced by an agent in a subject means that the subject, in the parameter or parameters being tested, is more similar to other subjects in which the agent does not produce a significant anticancer effect than to other subjects in which the agent does produce a significant anticancer effect.

Pharmaceutical compositions can be used in the preparation of individual, single unit dosage forms. Pharmaceutical compositions and dosage forms provided herein comprise an immunomodulatory compound provided herein, or a pharmaceutically acceptable salt, solvate, hydrate, stereoisomer, or prodrug thereof. Pharmaceutical compositions and dosage forms provided herein can further comprise one or more excipients.

Pharmaceutical compositions and dosage forms provided herein can also comprise one or more second active ingredients. Consequently, pharmaceutical compositions and dosage forms provided herein comprise the active ingredients disclosed herein (e.g., an immunomodulatory compound). Examples of optional second, or additional, active ingredients are disclosed herein.

Kits and compositions for carrying out the methods provided herein are also contemplated. In certain embodiments, provided herein are kits useful for determining the efficacy of an anti-cancer compound. In certain embodiments, provided herein are kits useful for assessing the efficacy of a compound in treating a patient. In some embodiments, provided herein are kits useful for determining the effect of an immunomodulatory compound. The kit comprises a solid support, and a means for detecting the gene, protein, or glycoprotein expression of at least one biomarker in a biological sample. Such a kit may employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The biological sample can be, for example, a cell culture, a cell line, a tissue, an oral tissue, gastrointestinal tissue, an organ, an organelle, a biological fluid, a blood sample, a urine sample, or a skin sample.

In one embodiment a kit provided herein comprises an illudin compound provided herein, or a pharmaceutically acceptable salt, solvate or hydrate thereof. Kits may further comprise additional active agents, including but not limited to those disclosed herein.

### EXAMPLES

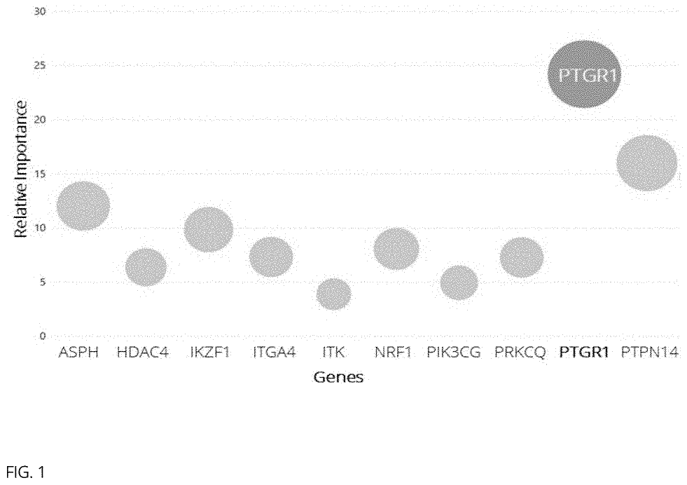

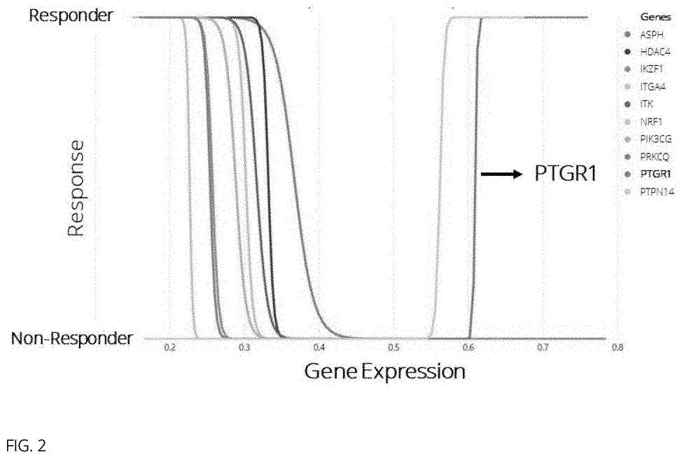

Certain embodiments provided herein are illustrated by the following non-limiting example. As shown in Example 1, analyses of in vitro Illudin (LP-184) sensitivity data from a panel of cancer cell lines with known multi-omic molecular profiles yielded a signature of 10 genes (ASPH, HDAC4, IKZF1, ITGA4, ITK, NRF1, PIK3CG, PRKCQ, PTGR1, and PTPN14) capable of predicting illudin sensitivity with up to or including 100% accuracy on blind testing. The gene expression, DNA mutation, DNA methylation and protein expression status of these 10 genes was further computed as a correlation with Illudin (LP-184) sensitivity. Out of these 10 genes, 3 genes emerged as having highly significant correlations with Illudin bioactivity at the multiomic level, as represented in table 1. These gene identities are PTGR1, PTPN14 and ASPH. These 3 genes also carry the highest gene weightage in terms of relative importance while determining Illudin (LP-184) sensitivity according to FIG. 1. They were revealed to be the most prominent genes in classifying a sample as an illudin responder or non-responder as shown in FIG. 2. Overall, a novel gene set that determines sensitivity to and predicts responses to Illudins (LP-184) in cancer has been established.

### Example 1

Genes Correlated With LP-184 (Illudin Family) Sensitivity in Cancer

| Gene name | Parameter | Correlation coefficient | p value |
|---|---|---|---|
| PTGR1 | Gene expression | 0.868 | 0 |
| | DNA methylation | −0.728 | 0 |
| | Protein expression | 0.561 | 0.000004 |
| PTPN14 | Gene expression | 0.709 | 0 |
| | DNA methylation | −0.639 | 0 |
| ASPH | Gene expression | 0.536 | 0.000012 |
| | DNA mutation | −0.343 | 0.007733 |
| | Protein expression | 0.579 | 0.000002 |

FIG. 1 shows the relative importance of an exemplary gene signature for hydroxyureamethylacylfulvene. In this relative variable importance graph, gene weightage analysis was performed to analyze the relative ranking of the top 10 genes in the hydroxyureamethylacylfulvene signature in cancer. The data shows that PTGR1 (Prostaglandin Reductase 1) followed by PTPN14 and ASPH are gene signatures for hydroxyureamethylacylfulvene sensitivity.

FIG. 2. shows PTGR1 and PTPN14 association with positive response to hydroxyureamethylacylfulvene. In this sensitivity analysis graph, the effect of gene expression on the response variable is studied across LP-184 gene signatures using the lekprofile function. High expression of PTGR1 (Prostaglandin Reductase 1) was correlated to positive response to LP-184) with PTPN14 being the next most highly correlated gene. This experiment was conducted on a panel of cancer cells.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the embodiments and are encompassed by the appended claims.

1. A method of treating solid tumor cancer in a human subject with a targeted drug therapy comprising:
extracting a biological sample from the human subject;
measuring the level of expression of PTGR1, PTPN14, ASPH or combination thereof in the biological sample;
identifying a patient having cancer sensitive to treatment with an illudin-based anti-cancer agent;
treating the human subject with the illudin-based anti-cancer agent having the following structure: 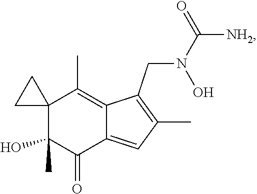 [SMILES: CC1=C(CN(O)C(N)=O)C2=C(C)C3(CC3)[C@@](C)(O)C(=O)C2=C1]
wherein the expression level in the human subject of PTGR1, PTPN14, or ASPH is greater than the level in human without the solid tumor cancer.

2. The method of claim 1, wherein the solid tumor cancer is prostate cancer, ovarian cancer, kidney cancer, and thyroid cancer.

3. The method of claim 1, wherein the solid tumor cancer is colorectal cancer, pancreatic cancer, primary liver cancers, kidney cancer, ovarian cancer, uterine cancer, or breast cancer.

4. The method of claim 1, wherein the ASPH is methylated.

5. A method of treating solid tumor cancer with a targeted drug therapy comprising:
identifying a patient having cancer sensitive to treatment with a compound of the formula: 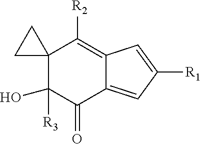
wherein R1, R2 and R3 are independently (C1-C4) alkyl, methyl, or hydroxyl
measuring the level of expression of PTGR1 of a combination thereof in the biological sample.

6. The method of claim 5, further comprising the step of measuring the level of expression of PTGR1 of a combination thereof in the biological sample.

7. The method of claim 5, further comprising the step of measuring the level of expression of PTPN14 in the biological sample.

8. The method of claim 5, further comprising e step of measuring the level of expression of ASPH in the biological sample.

9. The method of claim 5, wherein the cancer is newly diagnosed, relapsed, or refractory.

10. The method of claim 5, further comprising modifying a targeted drug therapy based on expression of a gene.

11. The method of claim 9, wherein ASPH is methylated.

12. The method of claim 1, wherein the cancer is colorectal cancer, pancreatic cancer, primary liver cancers, kidney cancer, ovarian cancer, uterine cancer, lung cancer, breast cancer, prostate cancer, sarcomas, or adipose tissue cancer.

13. A method for predicting response lent in a patient having a solid tumor, comprising:
obtaining a biological sample from the patient;
measuring the level of expression of PTGR1, PTPN 14, ASPH or a combination thereof in the biological sample; and
comparing the level of expression in the biological sample to that of a biological sample from a subject not having a solid cancer;
wherein an increased level of the expression in the biological sample from the patient relative to that from the subject not having the solid tumor indicates a likelihood of an effective response to the treatment with a compound of the formula: 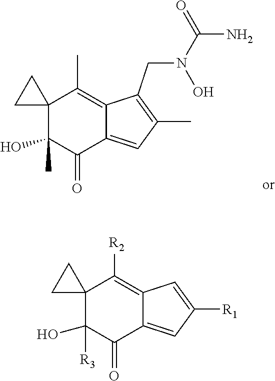
or a pharmaceutically acceptable salt, solvate or stereoisomer thereof.

14. A kit for determining whether an anti-cancer effect is likely to be produced in a cancer by an illudin, comprising a means for determining the level of PTGR1, PTPN14, ASPH or a combination thereof expression in a cancer relative to a normal value from biological sample from the patient relative to that from the subject having the solid tumor.

15. The method of claim 16, wherein the cancer is colorectal cancer, pancreatic cancer, primary liver cancers, kidney cancer, ovarian cancer, uterine cancer, lung cancer, breast cancer, prostate cancer, sarcomas, or adipose tissue cancer.

16. The kit of claim 18, wherein the illudin has 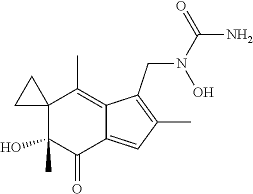 [SMILES: CC1=C(CN(O)C(N)=O)C2=C(C)C3(CC3)[C@@](C)(O)C(=O)C2=C1]
